SEQUENCE LISTING (2 sequences)

SEQ ID NO: 1 - protein, 2509 aa, Homo sapiens [sequence omitted]

SEQ ID NO: 2 - protein, 2511 aa, homo sapiens
MEEVVIAGMSGKLPESENLQEFWDNLIGGVDMVTDDDRRWKAGLYGLPRRSGKLKDLSRFDASFFGVHPKQAHTMDPQLRLLLEVTYEAIVDGGINPDSLRGTHTGVWVGVSGSETSEALSRDPETLVGYSMVGCQRAMMANRLSFFFDFRGPSIALDTACSSSLMALQNAYQAIHSGQCPAAIVGGINVLLKPNTSVQFLRLGMLSPEGTCKAFDTAGNGYCRSEGVVAVLLTKKSLARRVYATILNAGTNTDGFKEQGVTFPSGDIQEQLIRSLYQSAGVAPESFEYIEAHGTGTKVGDPQELNGITRALCATRQEPLLIGSTKSNMGHPEPASGLAALAKVLLSLEHGLWAPNLHFHSPNPEIPALLDGRLQVVDQPLPVRGGNVGINSFGFGGSNVHIILRPNTQPPPAPAPHATLPRLLRASGRTPEAVQKLLEQGLRHSQDLAFLSMLNDIAAVPATAMPFRGYAVLGGERGGPEVQQVPAGERPLWFICSGMGTQWRGMGLSLMRLDRFRDSILRSDEAVKPFGLKVSQLLLSTDESTFDDIVHSFVSLTAIQIGLIDLLSCMGLRPDGIVGHSLGEVACGYADGCLSQEEAVLAAYWRGQCIKEAHLPPGAMAAVGLSWEECKQRCPPGVVPACHNSKDTVTISGPQAPVFEFVEQLRKEGVFAKEVRTGGMAFHSYFMEAIAPPLLQELKKVIREPKPRSARWLSTSIPEAQWHSSLARTSSAEYNVNNLVSPVLFQEALWHVPEHAVVLEIAPHALLQAVLKRGLKPSCTIIPLMKKDHRDNLEFFLAGIGRLHLSGIDANPNALFPPVEFPAPRGTPLISPLIKWDHSLAWDVPAAEDFPNGSGSPSAAIYNIDTSSESPDHYLVDHTLDGRVLFPATGYLSIVWKTLARALGLGVEQLPVVFEDVVLHQATILPKTGTVSLEVRLLEASRAFEVSENGNLVVSGKVYQWDDPDPRLFDHPESPTPNPTEPLFLAQAEVYKELRLRGYDYGPHFQGILEASLEGDSGRLLWKDNWVSFMDTMLQMSILGSAKHGLYLPTRVTAIHIDPATHRQKLYTLQDKAQVADVVVSRWLRVTVAGGVHISGLHTESAPRRQQEQQVPILEKFCFTPHTEEGCLSERAALQEELQLCKGLVQALQTKVTQQGLKMVVPGLDGAQIPRDPSQQELPRLLSAACRLQLNGNLQLELAQVLAQERPKLPEDPLLSGLLDSPALKACLDTAVENMPSLKMKVVEVLAGHGHLYSRIPGLLSPHPLLQLSYTATDRHPQALEAAQAELQQHDVAQGQWDPADPAPSALGSADLLVCNCAVAALGDPASALSNMVAALREGGFLLLHTLLRGHPLGDIVAFLTSTEPQYGQGILSQDAWESLFSRVSLRLVGLKKSFYGSTLFLCRRPTPQDSPIFLPVDDTSFRWVESLKGILADEDSSRPVWLKAINCATSGVVGLVNCLRREPGGNRLRCVLLSNLSSTSHVPEVDPGSAELQKVLQGDLVMNVYRDGAWGAFRHFLLEEDKPEEPTAHAFVSTLTRGDLSSIRWVCSSLRHAQPTCPGAQLCTVYYASLNFRDIMLATGKLSPDAIPGKWTSQDSLLGMEFSGRDASGKRVMGLVPAKGLATSVLLSPDFLWDVPSNWTLEEAASVPVVYSTAYYALVVRGRVRPGETLLIHSGSGGVGQAAIAIALSLGCRVFTTVGSAEKRAYLQARFPQLDSTSFANSRDTSFEQHVLWHTGGKGVDLVLNSLAEEKLQASVRCLATHGRFLEIGKFDLSQNHPLGMAIFLKNVTFHGVLLDAFFNESSADWREVWALVQAGIRDGVVRPLKCTVFHGAQVEDAFRYMAQGKHIGKVVVQVLAEEPEAVLKGAKPKLMSAISKTFCPAHKSYIIAGGLGGFGLELAQWLIQRGVQKLVLTSRSGIRTGYQAKQVRRWRRQGVQVQVSTSNISSLEGARGLIAEAAQLGPVGGVFNLAVVLRDGLLENQTPEFFQDVCKPKYSGTLNLDRVTREACPELDYFVVFSSVSCGRGNAGQSNYGFANSAMERICEKRRHEGLPGLAVQWGAIGDVGILVETMSTNDTIVSGTLPQRMASCLEVLDLFLNQPHMVLSSFVLAEKAAAYRDRDSQRDLVEAVAHILGIRDLAAVNLDSSLADLGLDSLMSVEVRQTLERELNLVLSVREVRQLTLRKLQELSSKADEASELACPTPKEDGLAQQQTQLNLRSLLVNPEGPTLMRLNSVQSSERPLFLVHPIEGSTTVFHSLASRLSIPTYGLQCTRAAPLDSIHSLAAYYIDCIRQVQPEGPYRVAGYSYGACVAFEMCSQLQAQQSPAPTHNSLFLFDGSPTYVLAYTQSYRAKLTPGCEAEAETEAICFFVQQFTDMEHNRVLEALLPLKGLEERVAAAVDLIIKSHQGLDRQELSFAARSFYYKLRAAEQYTPKAKYHGNVMLLRAKTGGAYGEDLGADYNLSQVCDGKVSVHVIEGDHRTLLEGSGLESIISIIHSSLAEPRVSVREG

(19) United States
(12) Patent Application Publication
(10) Pub. No.: US 2011/0124021 A1
(43) Pub. Date: May 26, 2011

(54) PHOSPHORYLATED FATTY ACID SYNTHASE AND CANCER

(76) Inventor: Susan Medghalchi, Ellicott City, MD (US)

(21) Appl. No.: 12/742,910

(22) PCT Filed: Nov. 13, 2008
(86) PCT No.: PCT/US2008/083456

§ 371 Date: Feb. 3, 2011

Related U.S. Application Data
(60) Provisional application No. 60/987,763, filed on Nov. 13, 2007.

(51) Int. Cl. C12Q 1/48; C07K 16/40

(52) U.S. Cl. 435/15; 530/389.8; 530/388.26

(57) ABSTRACT

The disclosed invention relates to the detection of phosphorylated fatty acid synthase as a diagnostic and a component in the identification and treatment of cancer. The disclosed methods permit early and accurate diagnosis of cancer to enable more effective therapy and to enhance patient survival and quality of life.

### RELATED APPLICATIONS

This application claims benefit of priority from U.S. Provisional Patent Application 60/987,763, filed Nov. 13, 2007, and is a U.S. national phase application of International (PCT) Application No. PCT/US2008/083456 filed Nov. 13, 2008, which applications are hereby incorporated by reference as if fully set forth.

### FIELD OF THE DISCLOSURE

This disclosure relates to the early and accurate diagnosis of cancer to enable more effective therapy and to enhance patient survival and quality of life. This disclosure provides an assay for cancer based upon the identification of fatty acid synthase (FAS), particularly in a phosphorylated form, to improve detection methods for the presence, course, and treatment of cancer.

### BACKGROUND OF THE DISCLOSURE

During the last decade, increasing interest has developed in fatty acid synthase (FAS) as a potential diagnostic and therapeutic target for human cancer. These notions are based on two observations: FAS is highly expressed in most common human cancers, and pharmacological inhibition of FAS leads to apoptosis of human cancer cells in vitro and in vivo.

For example, Table 1 illustrates the expression of FAS in a number of human cancers.

TABLE 1

| | Precursor Lesion | Infiltrating Cancer | Normal Tissue | Prognostic Impact |
|---|---|---|---|---|
| Breast (16-21) | ↑↑ in situ cancer | ↑↑↑↑ to [symbol] present in | ↑↑ lactation, | >4-fold risk of death if expressed in Stage I disease |
| | | serum | ↑apocrine changes | |
| Prostate (10, | ↑↑↑ in prostatic | ↑↑↑ to [symbol] . Persists | ↑ to [symbol] | >4-fold risk of disease progression, increased risk of |
| 22-29) | intraepithelian | following androgen | | non-organ confined disease |
| | neoplastia (PIN | ablation. FAS copy | | |
| | | number gain. | | |
| Colon (30-32) | ↑↑ in adenomas | ↑↑↑ to ↑↑ present in serum | [symbol] | None detected |
| Stomach (33) | ↑ in adenomas | ↑↑ in cancer | [symbol] | None detected |
| Lung (34-36) | ↑↑ in dysplasia | ↑↑↑ in squamous ca. | Some expression in | Associated with low-stage aggressive disease |
| | | ↑↑ in adenoca present in | type II pneuomocytes | |
| | | serum | | |
| Mesothelioma | Precurson lesion | ↑↑↑ in about 50% | [symbol] | None detected |
| (37) | not defined | | | |
| Oral Cavity & | ↑↑ in dysplasia | ↑↑↑ in cancer | ↑ in normal tissue | None detected |
| Esophagus | | | near cancer | |
| (38-40) | | | | |
| Head & Neck | Not studied | ↑↑ along with HER2/neu | [symbol] | Increased in well differentiated tumor |
| squamous | | | | |
| cancer (41) | | | | |
| Ovary (19, 42, | Precursor lesion not | ↑↑↑to [symbol] present in serum | [symbol] | Increased levels in poorly differentiated cancer |
| 43) | defined | | | |
| Melanoma | ↑in situ melanoma | ↑↑↑ infiltrating metastases | [symbol] | Correlates with thickness of lesion. Increased in |
| (skin) (39) (44) | | | | atypical Spitz nevi. |
| Endometrium | Not studied | ↑↑↑ to [symbol] | ↑↑↑ in proliferative | Increased levels in poorly differentiated cancer. |
| (45-47) | | | endometrium | Associated with recurrence |
| Thyroid (48) | Not studied | ↑↑↑ in high grade cancer | [symbol] | Increase levels in poorly differentiated cancer |
| Parathyroid (49) | ↑↑ in adenoma | Not studied | ↑↑in hyperplasia | None detected |
| Kidney (50) | Not studied | ↑↑ in renal cell carcinoma | Some expression in | None detected |
| | | and urothelial carcinoma | distal tubules | |
| Urinary Bladder | Not studied | ↑↑ in carcinoma FAS | [symbol] | Correlates with recurrence |
| (51, 52) | | activity increased | | |
| Retinoblastoma | Not studied | ↑↑ in cancer | [symbol] | Correlates with extent of disease |
| Nephroblastoma | Not studied | ↑↑ in cancer | Some expression in | Correlates with survival & recurrence |
| (Wilm's tumor) | | | distal tubules | |
| (54) | | | | |
| Soft tissue | Not studied | ↑↑ most common in MFH | [symbol] | Associated with poor prognosis |
| sarcomas (55) | | | | |

FAS is a complex, multifunctional enzyme that contains seven catalytic domains and a 4′-phosphopantetheine prosthetic group on a single polypeptide with a relative molecular weight of about 270 kDa (Smith, FASEB J, 8: 1248-1259 (1994); Wakil, Biochem., 28: 4523-4530 (1989)). The human FAS amino acid sequence is known and deposited with accession number AAA73576 (with a length of 2509 amino acid residues) and NP_{—}004095 (with a length of 2511 amino acid residues).

FAS is the sole mammalian enzyme that catalyzes the NADPH dependent condensation of malonyl-CoA and acetyl-CoA to produce the 16-carbon saturated free fatty acid palmitate (see FIG. 1). Although FAS performs the de novo synthesis of fatty acid from carbohydrates, the immediate proximal enzyme in the pathway, acetyl-CoA carboxylase (ACC) is the rate-limiting enzyme of fatty acid synthesis (Ruderman et al., Am. J. Physiol., 276: E1-E18 (1999))

The citation of documents herein is not to be construed as reflecting an admission that any is relevant prior art. Moreover, their citation is not an indication of a search for relevant disclosures. All statements regarding the dates or contents of the documents is based on available information and is not an admission as to their accuracy or correctness.

### BRIEF SUMMARY OF THE DISCLOSURE

The disclosure relates to detecting or measuring expression of phosphorylated fatty acid synthase (FAS) as an indicator, or marker, of the presence of cancer in a subject. The disclosure is based in part on the observation that FAS has been observed at high (above normal) levels in most common cancers, including colon, lung, prostate and breast cancers. The disclosure is also based in part on detecting phosphorylated FAS produced by cancer cells in comparison to non-phosphorylated FAS produced by non-cancer, or normal, cells. In a first aspect, the disclosure provides methods and compositions for detecting or measuring expression of a phosphorylated FAS polypeptide (containing one or more phosphorylated amino acid residues) or a fragment of the FAS polypeptide containing one or more phosphorylated residues. In some embodiments, the methods and compositions include a complex comprising a binding agent which specifically binds the phosphorylated FAS polypeptide, or phosphorylated fragment thereof, to form a detectable complex. The specificity of the binding agent may be such that the agent detectably binds the phosphorylated FAS polypeptide, or phosphorylated fragment thereof, to the exclusion of the counterpart non-phosphorylated polypeptide or fragment. In some cases, the binding agent is an antibody, such as a monoclonal antibody.

In a second aspect, the disclosure provides a method of identifying the presence of cancer in a subject based on the presence of a phosphorylated FAS polypeptide, or phosphorylated fragment thereof, in a biological sample from the subject. The method may comprise detecting or measuring the presence of a phosphorylated FAS polypeptide, or a phosphorylated fragment of the polypeptide, in a biological sample obtained from a subject. In some embodiments, the method may be used as a screening method or assay to identify individuals afflicted with cancer. In other embodiments, the method may be used to confirm a diagnosis of the presence of cancer, such as in combination with one or more other diagnostic methods or protocols.

In an additional aspect, the disclosure provides a method of selecting subjects with phosphorylated FAS for treatment with FAS targeted therapies. In some embodiments, the method may be used to select or identify a subject as having tumor cells expressing phosphorylated FAS and then administering a FAS targeting treatment to the subject, optionally in combination with one or more other treatments against the tumor cells. In some cases, the method may comprise detecting the presence of a phosphorylated FAS polypeptide, or a phosphorylated fragment thereof, in a biological sample obtained from a subject and administering a FAS inhibitor to the subject.

In a further aspect, the disclosure provides a method of detecting or measuring disease progression, or efficacy of treatment, based on the expression of a phosphorylated FAS polypeptide, or a phosphorylated fragment thereof. The method may comprise measuring the level of a phosphorylated FAS polypeptide, or a phosphorylated fragment thereof, in a biological sample from said subject; and repeating the measuring over time. An increase or decrease in the level of expression over time indicates an increase or decrease, respectively, in tumor cells, or tumor cell activity. In some embodiments, the method may be used to monitor cancer or tumor cell burden. Optionally, the measurements over time are made before, during and/or following therapy to monitor the course of treatment and outcome for a subject.

In some embodiments, the methods and compositions of the disclosure are practiced in relation to specific cancers, such as cancers of the colon, lung, prostate, ovary and breast. In additional embodiments, the methods and compositions are practiced in relation to human subjects and patients.

In further embodiments, the methods and compositions of the disclosure are based on the detection of a phosphorylated FAS polypeptide, or phosphorylated fragment thereof, in a blood, serum, or tumor cell containing sample. A number of phosphorylated serum proteins have been reported in humans, although phosphorylated FAS is not among them. A portion of human fetuin (α2-Heremans-Schmid protein) is phosphorylated on serine (Haglund et al., Biochem. J., 357: 437-445 (2001)) which may affect insulin signal transduction. C3 is phosphorylated by casein kinase released from platelets which enhances its binding to complement receptor 1 (Nilsson-Ekdahl and Nilsson, Eur. J. Immunol., 31: 1047-1054 (2001)). Other reported circulating phosphoproteins include cardiac troponin I and T (Labugger et al., Circulation 102: 1221-1226 (2000)), tumor type M2 pyruvate kinase (Luftner et al., Anticancer Res., 23: 991-997 (2003)), complement C3c (Goldknopf, et al., Biochem. Biophys. Res. Commun., 342: 1034-1039 (2006)), human prolactin (Oetting et al., J. Biol. Chem., 261: 1649-1652 (1986)), and neurofilament NF-H for which an ELISA assay was developed to monitor axonal injury (Shaw et al., Biochem. Biophys. Res. Commun., 336: 1268-1277 (2005)).

An additional aspect of the disclosure is a detectable complex comprising a phosphorylated FAS polypeptide, or a phosphorylated fragment of the polypeptide, and a binding agent. In some embodiments, the binding agent is specific for the phosphorylated, as opposed to the unphosphorylated, polypeptide or fragment thereof. In some embodiments, the complex includes a phosphorylated FAS polypeptide with a relative molecular weight (MW) of about 270 kiloDaltons (kDa). In some cases, the complex includes a polypeptide with a length of 2509 or 2511 amino acid residues. In other embodiments, the polypeptide may have the same relative MW but a length shorter than that of 2509 or 2511, such as by truncation or loss of one or a few amino acid residues from one or both ends of the FAS polypeptide. In some embodiments, the 2509 or 2511 residue polypeptide has the sequence represented by SEQ ID NO:1 or SEQ ID NO:2 as disclosed herein, respectively.

In alternative embodiments, the complex includes a phosphorylated fragment of a phosphorylated FAS polypeptide. In embodiments with an antibody as the binding agent, the fragment may have a length of at least five, or about five, amino acid residues that present a sufficient epitope for recognition by the antibody. Of course the fragment may be longer than five residues, up to one residue less than the full-length of a FAS polypeptide, so long as the fragment contains at least one phosphorylated residue.

Embodiments of the disclosure include those wherein the phosphorylated FAS polypeptide, or a phosphorylated fragment thereof, contains at least one phosphothreonine residue, at least one phosphoserine residue, and/or at least one phosphotyrosine residue.

While the present disclosure is described mainly in the context of human cancer, it may be practiced in the context of cancer of any animal. Preferred animals for the application of the present disclosure are mammals, particularly those important to agricultural applications (such as, but not limited to, cattle, sheep, horses, and other “farm animals”), animal models of cancer, and animals for human companionship (such as, but not limited to, dogs and cats).

### BRIEF DESCRIPTION OF THE FIGURES

FIG. 1 is a schematic of the fatty acid synthesis pathway.

FIG. 2 illustrates FAS expression in prostate (A), colon (B), and breast cancer (C).

FIG. 3 shows a representative FAS ELISA standard curve with human FAS.

FIG. 4 illustrates FAS levels in cancer and normal subjects.

FIG. 5 illustrates FAS expression in normal human and human cancer cell lines.

FIGS. 6A and 6B, illustrates FAS from human cancer cells is phosphorylated on Thr/Pro.

FIGS. 7A and 7B, illustrates that phosphorylated FAS increases with okadaic acid treatment (part A) while total FAS protein is reduced (part B).

FIG. 8 shows the sequence of SEQ ID NO:1, with 2509 amino acid residues, and 7 threonine-proline dimers at positions 827, 976, 1406, 1982, 2202, 2354, or 2432. One dimer is within the acyl-carrier protein domain of FAS, which extends from residue 2117-2205 (Joshi et al., J. Biol. Chem., 278: 33142-33149 (2003)). Additionally, the serine at position 2154 is the site of the 4′-phosphopantetheine prosthetic group (Joshi et al., ibid.) which is not a “phosphorylated residue” as the term is used herein.

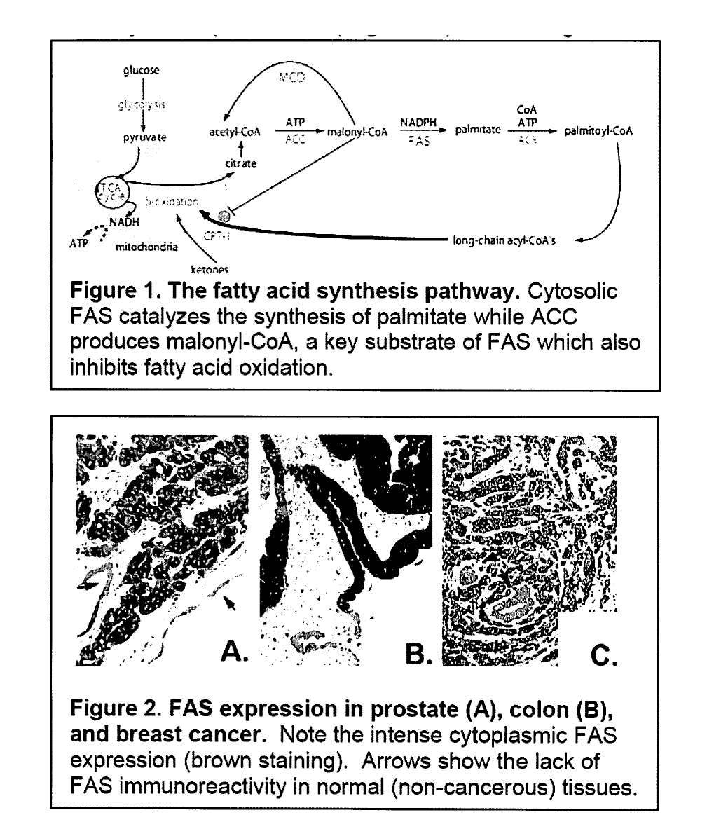

### DETAILED DESCRIPTION OF MODES OF PRACTICING THE DISCLOSURE

### General

FAS is the enzyme which catalyzes the de novo synthesis of fatty acids predominantly from dietary carbohydrates. In addition to its expression in human cancers, it has been observed that FAS circulates at high (above normal) levels in the blood of colon, breast, lung, ovarian, and prostate cancer patients as compared to normal (cancer-free) subjects. Moreover, increased FAS expression is associated with aggressive disease in breast, prostate, ovary, endometrium, urinary bladder, pediatric malignancies, and soft tissue sarcomas. FIG. 2 illustrates the high levels of FAS expression in prostate, colon, and breast carcinomas with immunohistochemistry.

In contrast, normal human tissues do not readily undergo lipogenesis (Weiss et al., Biol. Chem. Hoppe Seyler; 367:905-912 (1986)) and FAS expression is largely restricted to proliferative endometrium (Pizer et al., Cancer, 83: 528-537 (1998)), deciduas (Pizer et al., Int. J. Gynecol. Pathol, 16: 45-51 (1997)), lactating breast (Smith et al., J. Natl. Cancer Inst., 73: 323-329 (1984), Thompson et al., Pediatr. Res., 19: 139-143 (1985)), Type II pneumocytes (Ridsdale and Post, Am. J. Physiol. Lung Cell. Mol. Physiol., 287: L743-751 (2004)) and neurons (Kim et al., Am. J. Physiol. Endocrinol. Metab., 283: E867-879 (2002), Landree et al., J. Biol. Chem., 279: 3817-3827 (2004)). Although FAS does not contain the carboxyterminal di-lysine motif (KKXX or KXKXX) common to secretory proteins (Jackson et al., J. Cell Biol., 121: 317-333 (1993), Duden, Mol. Membr. Biol., 20: 197-207 (2003)), FAS is secreted in milk as part of the milk fat globule complex (Keon et al., Int. J. Biochem., 25: 533-543 (1993)).

FAS circulates in the blood of breast, prostate, colon, and ovarian cancer patients at levels significantly higher than normal subjects (Wang et al., Cancer Lett., 167: 99-104 (2001), Wang et al., Clin. Chico. Acta., 304: 107-115 (2001)). It has been reported (Wang et al., ibid.) that a comparison of serum FAS levels for 25 healthy normal subjects (15 women and 10 men, ages 28-60) with levels found in pre-treatment sera from patients with breast cancer (N=30), prostate cancer (N=29), ovarian cancer (N=30), and colon cancer (N=30) observed that FAS concentrations in the patient sera were significantly higher than those of the healthy controls (p<0.01 for each tumor type). When a cutoff concentration equal to the mean concentration for healthy controls+2 SD (standard deviations) was selected (i.e. specificity of approximately 95%), positive detection rates were 83% (breast), 53% (prostate), 90% (colon) and 40% (ovarian). The FAS assay used had within and between-run CVs (coefficient of variation) of less than 10% with recoveries of >92.4% (Wang et al., ibid.).

Similar results were reported with a monoclonal/monoclonal FAS assay with elevation of FAS in breast cancer patients and no correlation with the breast cancer marker CA27.29 (Wang et al., J. Immunoassay Immunochem. 23: 279-292 (2002), Wang et al., J. Exp. Ther. Oncol., 4: 101-110 (2004)). Moreover, in breast cancer patients, circulating FAS levels correlated positively with tumor stage (Wang et al., Cancer Lett., 167: 99-104 (2001)).

Additionally, FAS derived from tumor cell lines is phosphorylated on threonine residues while FAS from non-transformed cells is not phosphorylated. Moreover, a study on FAS expression and activity in normal murine mammary cells compared to mouse mammary tumors induced by either rodent polyoma (Py) virus or murine mammary tumor virus (MMTV) reported differences in specific activity of FAS among the tumor cell lines (Hennigar et al., Biochim Biophys Acta, 1392: 85-100 (1998)). Phosphorylation of FAS in the tumor cell lines was investigated as a potential cause of the differences in enzyme activity. Using immunoprecipitation with anti-FAS followed by immunoblots with anti-phosphoserine and anti-phosphothreonine antibodies, phosphorylation on both serine and threonine residues of FAS was restricted to the mammary tumor cell lines. FAS from normal murine mammary cells was not phosphorylated.

SKBR3 human breast cancer cells were also studied and reported to be phosphorylated on serine, threonine and tyrosine residues. In contrast, earlier reports of normal tissues demonstrated that FAS purified from rat liver and adipose tissue was not phosphorylated (Rous, FEBS Lett., 44: 55-58 (1974), Ramakrishna and Benjamin, Prep. Biochem., 13: 475-488 (1983)). This was consistent with the reports on the liver that fatty acid synthesis pathway activity is regulated by phosphorylation of acetyl-CoA carboxylase (ACC) and not by phosphorylation of FAS (Ruderman et al., Am. J. Physiol., 276: E1-E18 (1999)).

These observations, in part, led to the instant disclosure's description of methods and compositions based on phosphorylated FAS.

Methods and Assays

As described herein, the disclosure includes methods and compositions for detecting or measuring the expression level of phosphorylated FAS polypeptide, or a fragment thereof, where the polypeptide or fragment contains one or more phosphorylated amino acid residues distinct from the 4′-phosphopantetheine prosthetic group found on FAS polypeptides.

Embodiments of the disclosure include a FAS polypeptide, or fragment thereof, containing one or more of a phosphothreonine, phosphoserine, or phosphotyrosine residues. More specifically, the disclosure includes detecting or measuring a phosphorylated FAS polypeptide, or fragment thereof, as it may be present in a subject, such as a human patient. In many embodiments, the detection or measurement is used in cases of elevated FAS levels, such as that observed in obese subjects.

In some embodiments, a method of the disclosure may be used to qualitatively detect the presence of a phosphorylated FAS polypeptide, or fragment thereof. In some cases, such a method may be used to determine whether the polypeptide or fragment is present or not. In other embodiments, a method may be used to qualitatively measure the amount of the polypeptide or fragment. In some cases, such a method may be used to determine the expression level of the polypeptide or fragment, and optionally provide a measurement in the form of amount per volume like nanogram of polypeptide or fragment per milliliter of volume. Alternatively, a measurement may be based on the number of molecules per volume, like one expressed in terms of molarity.

In many embodiments, the methods of the disclosure are practiced with the use of a biological sample from a subject, such as a fluid or cell containing sample from a human patient. In some cases, the fluid sample may be a blood, plasma, or serum sample. Non-limiting methods of the disclosure include determinations of phosphorylated FAS polypeptide, or a fragment thereof, in amounts based on ng/ml, such as above 4 ng/ml or above 10 ng/ml.

In other embodiments, a method may be practiced with the use of a cell containing sample, or extract thereof, obtained from a subject. In some cases, the sample may contain tumor cells, and use of the sample in a method of the disclosure may be used to confirm a determination of the presence of tumor cells or cancer. In other cases, the sample may be from a subject suspected as having cancer, and so the sample is suspected to contain tumor cells. Non-limiting embodiments of the disclosure include the use of such a sample to determine, or diagnose, the presence of tumor cells in the sample and so cancer in the subject.

The detection or measurement of a phosphorylated FAS polypeptide, or a fragment thereof, in a sample may be made directly or indirectly. A non-limiting example of a direct method is with Pro-Q® Diamond gel stain (Molecular Probes) and analysis using a Typhoon 940 Laser Scanner. In many embodiments, an indirect method is used, such as via detection or measurement of a complex containing the polypeptide or fragment bound to a binding agent that specifically recognizes the polypeptide or fragment. Thus the binding agent binds to the phosphorylated, but not the non-phosphorylated, form of the polypeptide or fragment, and so may also be termed a detection agent.

In many embodiments, the binding agent is an antibody or antigen binding fragment thereof. Non-limiting examples include the use of and Fᵥ or F_{ab} fragment of an antibody that specifically binds a phosphorylated FAS polypeptide, or fragment thereof, as described herein. In some cases, the antibody is a monoclonal antibody that binds in part, a phosphorylated residue in the FAS polypeptide or fragment thereof. Thus the antibody may be referred to as a detection antibody. In other embodiments, a polyclonal antibody, or a combination of monoclonal antibodies, may be used. In many embodiments, the binding agent is detectably labeled to facilitate the detection of the phosphorylated FAS polypeptide or fragment thereof.

The detection antibody may itself be a “primary antibody” which is unlabeled and so detected by binding of one or more detectably labeled secondary antibodies that recognize the primary antibody. In some embodiments, the label is an enzyme that produces a detectable signal by catalyzing a reaction with a substrate. Non-limiting examples include the use of horseradish peroxidase (HRP) or alkaline phosphatase (AP).

As described herein, a complex of the disclosure comprises the detection agent and a phosphorylated FAS polypeptide or fragment thereof. In some embodiments, the complex further comprises an additional binding agent which immobilizes, or captures, either the detection agent or the phosphorylated FAS polypeptide, or fragment thereof. The immobilization, or capture, may be mediated by the immobilization of the additional binding agent on a solid phase substrate, such as the surface of a plastic or glass plate or a bead as non-limiting examples.

In some cases, the additional binding agent is also an antibody, termed a capture antibody, which binds the FAS polypeptide, or fragment thereof, in a manner that does not interfere with the interactions between the detection agent and the polypeptide or fragment thereof. A capture antibody of the disclosure may be a monoclonal or polyclonal antibody. Alternatively, it may be a combination, or cocktail, of monoclonal antibodies. In many cases, a capture antibody recognizes FAS polypeptides based upon a conserved or consensus sequence present even in FAS polypeptides with sequence in other regions of the molecule. The combination of detection and capture antibodies with a phosphorylated FAS polypeptide, or fragment thereof, may be termed a “sandwich” such that a method or process includes the formation and/or detection of the “sandwich” complex. Alternatives to a “sandwich” format include a competitive format, which may also be used in the disclosed methods and processes.

Embodiments of the disclosed methods include a diagnostic assay, such as an ELISA (enzyme-linked immunosorbent assay). In some cases, the assay is used to diagnose human cancer based on FAS phosphorylation. The assay includes the feature of differentiating the indication, provided by phosphorylated FAS in a biological sample from a subject, from normal FAS present from normal tissues, such as the liver. The differentiation may be of phosphorylated FAS polypeptide, or fragment thereof, from non-phosphorylated FAS. In some embodiments, the ability to differentiate is applied in cases of elevated FAS levels, such as that observed in obese subjects.

Of course the ELISA may be used in either a “sandwich” or competitive format. Alternatively, a competitive radioimmunosorbent assay (RIA) may also be used.

The methods and assays of the disclosure may be used to identifying the presence, or absence, of cancer (or tumor cells that express phosphorylated FAS) in a subject based on the presence, or absence, of a phosphorylated FAS polypeptide, or phosphorylated fragment thereof, in a biological sample from the subject. In some cases, the method or assay screens individuals, optionally asymptomatic, to identify those afflicted with, or free of, cancer or tumor cells that express phosphorylated FAS. In other cases, the method or assay may be used to confirm a diagnosis of the presence of cancer, such as in combination with one or more other diagnostic methods or protocols. In further cases, the method or assay is used to confirm a diagnosis of the absence of cancer, or tumor cells that express phosphorylated FAS. In yet additional cases, the cancer may be early stage and/or characterized by a pre-neoplastic lesion.

Non-limiting examples of cancers, or the tumor cells thereof, include cancer of the breast, prostate, colon, stomach, lung, mesothelium (mesothelioma), oral cavity, esophagus, head and neck (squamous cancer), ovary, pancreas, endometrium, thyroid, parathyroid, kidney, or urinary bladder; or a cancer selected from retinoblastoma, nephroblastoma (Wilm's tumor), or a soft tissue sarcoma.

In other embodiments, a subject identified as having cancer, or tumor cells, as described above, may be selected for treatment based upon the FAS expression phenotype.

A method may include selecting a subject identified as having cancer, or tumor cells, expressing phosphorylated FAS and then administering a FAS targeted treatment to the subject, optionally in combination with one or more other treatments against the cancer or tumor cells. Non-limiting examples of other treatments include surgery, radiation, and/or chemotherapy. In some cases, the method may comprise administering a FAS inhibitor to the subject. Non-limiting examples of a FAS inhibitor include C75 and C247.

Additional embodiments of the disclosure include a method or assay to detect or measure disease progression, or efficacy of treatment, over time based on the expression of a phosphorylated fatty acid synthase (FAS) polypeptide, or a phosphorylated fragment thereof. In many cases, the method or assay includes more than one detection or measurement over time for comparative purposes such that an increase or decrease in the level of expression over time indicates an increase or decrease, respectively, in tumor cells, or tumor cell activity, or cancer activity. In some embodiments, the method or assay may be used to monitor treatment progress or effectiveness when a FAS targeted therapy or other anti-cancer or anti-tumor therapy (as disclosed herein) is applied to a subject. Thus, the detections or measurements over time may be made before, during and/or following therapy to monitor the course of treatment and outcome for a subject.

In yet additional embodiments, the disclosure includes a method or process based on detecting or measuring FAS phosphorylation by immunohistochemistry (IHC). In some cases, the method may include the use of antibodies specific for phosphorylated FAS polypeptide, or a fragment thereof, to detect or measure expression of the phosphorylated form in a cell containing sample.

Phosphorylated FAS Polypeptides and Phosphorylated Fragments Thereof

As described herein, the disclosure includes the detecting or measuring of a full-length FAS protein, such as those with a relative MW of about 270 kDa, with one or more phosphorylated amino acid residues. FAS polypeptides with variations in the sequence, such as in the size of the full-length sequence as a non-limiting example, may be detected or measured in the practice of the disclosure based on the presence of the phosphorylated amino acid residue(s) in the polypeptide. Similarly, polypeptides with other sequence variations, such as those due to polymorphism as a non-limiting example, may also be detected or measured based on the phosphorylated amino acid residue. In some embodiments, the full-length FAS sequence may be 2509 or 2511 amino acids long. Alternatively, the lengths may be longer or shorter than 2509, or 2511, residues based on the size of the full length FAS polypeptide as present in a subject.

In embodiments of a fragment of the polypeptide, the size will necessarily be less than full-length, and so non-limiting examples include fragments of less than 2509 or 2511 residues as described herein. In some cases, the fragments will be fragments of SEQ ID NO:1 or 2 as described herein. Various fragments of these described sequences are recognized by the skilled person based upon knowledge in the field. In other cases, the fragments will simply be truncations of one or a few amino acid residues from one or both ends of a FAS polypeptide. In many instances, the fragments will continue to have a relative molecular weight of about 270 kDa.

Other fragments of a FAS polypeptide are disclosed herein or readily known to the skilled person in the field. For example, the sequences of SEQ ID NO:1 and 2 can be readily scanned for the presence of lysine or arginine residues to select tryptic digestion fragments of a FAS polypeptide. The digestion may be partial, to produce larger fragments, or to completion, where all seven threonine-proline sites and the 4′-phosphopantetheine prosthetic group would be on separate and distinct peptides. The seven threonine sites are residue 827, 976, 1406, 1982, 2202, 2354, or 2432 of SEQ ID NO:1 or the threonine sites at residue 827, 976, 1407, 1984, 2204, 2356, or 2434 of SEQ ID NO:2.

In alternative embodiments, FAS fragments produced by V8 protease, chymotrypsin, subtilisin, clostripain, endoproteinase Lys-C, endoproteinase Glu-C, endoproteinase Asp-N, and thermolysin may be used to generate additional fragments that can be detected by the methods and assays disclosed herein. Of course these fragments would contain one or more phosphorylated residues of FAS based on the location of the cleavage sites and the residue position(s).

Collectively, the fragments may be termed FAS phosphopeptides of the disclosure, which are at least five (5) amino acid residues in length (as found sequentially in a FAS polypeptide) to define an epitope recognized by a binding agent as described herein. As understood by the skilled person, the epitope need not be produced by five or more sequential residues but may instead be the result of protein folding to form an epitope from non-sequential residues. Additional embodiments include fragments of at least 10 or about 10, at least 15 or about 15, at least 20 or about 20, at least 25 or about 25, at least 30 or about 30, at least 35 or about 35, at least 40 or about 40, at least 45 or about 45, at least 50 or about 50, at least 75 or about 75, at least 100 or about 100, at least 200 or about 200, at least 300 or about 300, at least 400 or about 400, at least 500 or about 500, at least 750 or about 750, at least 1000 or about 1000, at least 1250 or about 1250, at least 1500 or about 1500, at least 1750 or about 1750, at least 2000 or about 2000, at least 2250 or about 2250, or at least 2500 or about 2500 sequential residues of a FAS polypeptide. Of course a fragment must contain one or more phosphorylated residue for use in the disclosed methods and assays. Non-limiting examples of a phosphorylated residue include a phosphothreonine residue, a phosphoserine residue, and at least one phosphotyrosine residue. Additional FAS phosphopeptides of the disclosure are those found in a biological sample of a subject, such as a human patient.

As described herein, a phosphorylated FAS polypeptide or fragment thereof may be present in a complex, such as a “sandwich” complex as disclosed herein. In some cases, the complex is immobilized on a solid phase substrate as described herein. In other embodiments, the complex is present in combination with other blood, serum, or plasma components, or other cellular components, present in the biological sample containing the complexed FAS polypeptide or fragment thereof.

### Additional Embodiments

The materials for use in the methods and assays of the present disclosure are ideally suited for preparation of kits produced in accordance with well known procedures. The disclosure thus provides kits comprising agents for the detection of expression of the disclosed phosphorylated FAS polypeptides and fragments. Such kits optionally comprise the agents with an identifying description or label or instructions relating to their use in the methods and assays of the present disclosure. Such a kit may comprise containers, each with one or more of the various reagents (typically in concentrated form) utilized in the methods and assays, including, for example, antibodies, buffers, wash solutions, etc. A set of instructions will also typically be included.

Having now generally provided the disclosure, the same will be more readily understood through reference to the following examples which are provided by way of illustration, and are not intended to be limiting of the disclosure, unless specified.

### EXAMPLES

### Example 1

### FAS Binding Antibodies and a Representative ELISA Format

FAS purified from human ZR-75-1 human breast cancer cells were used as an immunogen to produce hybridomas for reactivity to purified human FAS. FAS from other human or animal sources may also be used.

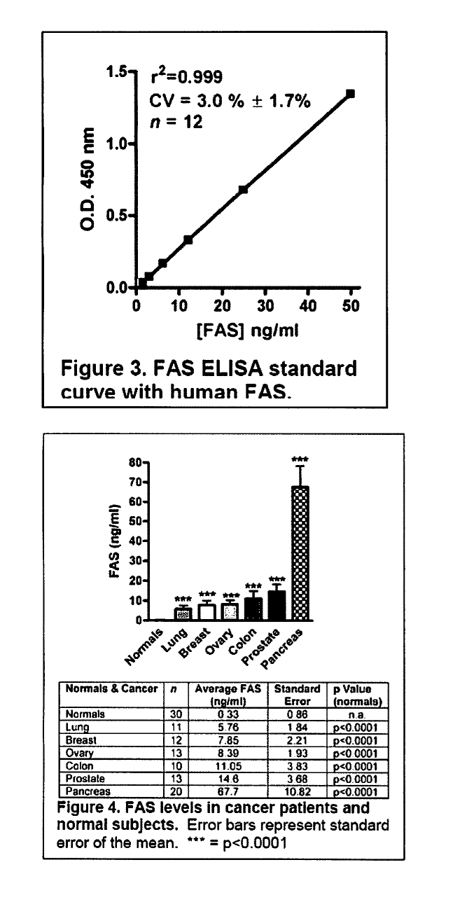

Two IgG1 producing clones were selected as capture and detection antibodies for a monoclonal/monoclonal ELISA assay through FAS epitope mapping. FIG. 3 is a representative standard curve summarizing within-plate standard curves with purified FAS. The assay is linear through FAS concentrations of 1.6-50 ng/ml with a CV of 3%. The day-to-day variability over 2 weeks with 8 assays generated a CV of 5.9%±1.9%. Analytical sensitivity is 0.301 ng/ml within 95% confidence using EP evaluator software.

### Example 2

### Elevated FAS Levels in Cancer Patient Sera

A FAS ELISA assay was used to confirm serological studies of FAS in cancer patients. Serum FAS levels were measured in 79 patients with active disease representing most common human cancer types and were compared to 30 male and female control subjects (that were clinically free of cancer). The results are shown in FIG. 4.

Average FAS levels ranged from a greater than 5-fold elevation in breast cancer patients to a greater than 67-fold elevation in pancreas cancer patients. The sensitivity for the detection of all cancers was 88.6% with a specificity of 86.7%. All patients with prostate, colon, and pancreas cancer had positive FAS values based on the normal average±2 standard deviations. Twelve (12) of 13 ovarian, 10 of 12 breast, and 6 of 11 lung cancer patients had positive FAS values. Thus, serum FAS levels are highly and significantly elevated in patients with common solid tumors.

### Example 3

### FAS is Phosphorylated in Cancer Cell Lines but not in Normal Cell Lines

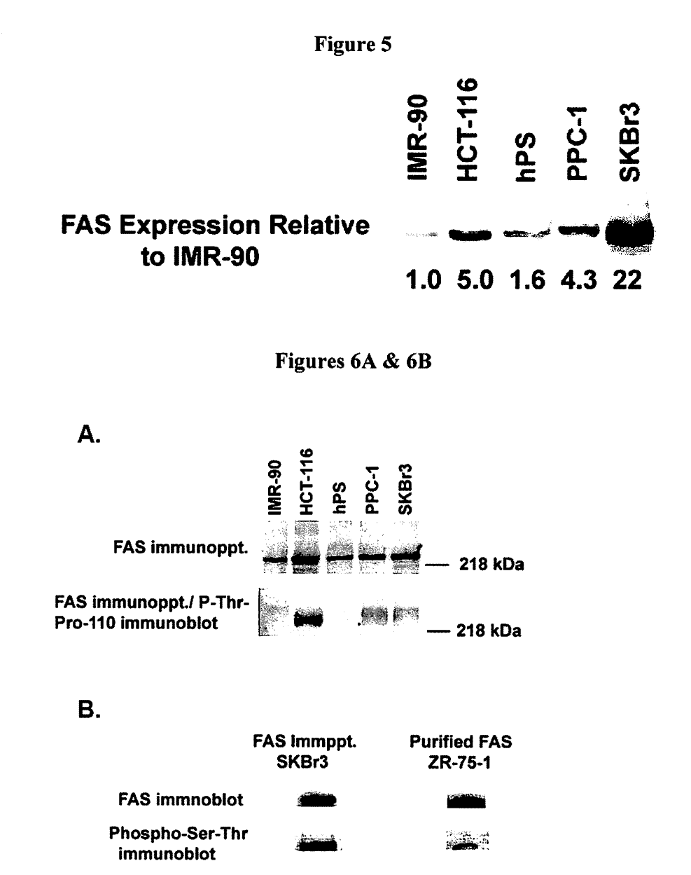

The relationship between FAS phosphorylation and cancer was investigated with a panel of the following human immortalized non-transformed cell lines: IMR-90 fetal lung, hPS human prostate and human cancer cell lines: HCT-116 colon, PPC-1 prostate, and SKBr3 breast. FAS expression was quantified by immunoblot as shown in FIG. 5. FAS enzyme levels (adjusted to total cellular protein) were quantitated by immunoblot and normalized to IMR-90. Both IMR-90 and hPS non-transformed cell lines had relatively low levels of FAS expression compared to the cancer cell lines, which ranged from 4.3 to 22 fold elevations compared to IMR-90 cells. The high level of FAS expression in SKBr3 cells is consistent with the observation that 28% of its cytosolic protein is FAS (Thompson et al., Biochem. Biophys Acta, 662: 125-130 (1981)).

Because the FAS expression levels in this panel of cells is consistent with observations made in vivo, the possibility of differential FAS phosphorylation in these cells was investigated. FIG. 6A illustrates the results from immunoprecipitating cellular contents, from the cell lines, with anti-FAS followed by immunoblotting of the immunoprecipitates with anti-FAS (upper panel) or anti-phosphothreonine-proline (lower panel) where the phosphothreonine requires an adjacent proline for antibody reactivity (Cell Signaling). All three tumor cell lines show evidence of FAS phosphorylation while the two non-transformed cell lines are negative. No immunoreactivity was detected with an anti-phosphotyrosine or an anti-phosphoserine antibody, although additional antibodies and FAS proteins were not tested. Additional human cancer cell lines, such as LnCAP, OVCAR-3, SKOV3 (ovary), RKO (colon), H460, LX7 (lung), CAPAN-1, and PANC-1 (pancreas) can also be assessed for differential FAS phosphorylation as described above.

Observations similar to those described above were obtained from short term (2 hours) labeling with ³²P in phosphate free medium. The pulse labeling does not favor incorporation of ³²P labeling of the 4′-phosphopantethiene prosthetic group on FAS via the CoA pool (data not shown).

FIG. 6B illustrates the results of SKBr3 cell material immunoblotted (labeled) with an anti-phosphosphoserine/threonine antibody preparation which confirms the phosphothreonine reactivity shown in FIG. 6A. Purified FAS from ZR-75-1 breast cancer cells, which were used as an immunogen for antibody production, is also immunolabeled by the antibody preparation.

### Example 4

### Confirmation of FAS Phosphorylation

FAS phosphorylation is also confirmed by using Pro-Q® Diamond gel stain (Molecular Probes) and analyzed using a Typhoon 940 Laser Scanner as a representative device. Determination of FAS phosphorylation by this alternative method provides an antibody-independent means to detect or measure FAS phosphorylation. Additionally, it is pointed out that the 4′-phosphopantethenylation of FAS precludes using metabolic labeling with ³²P to assess FAS phosphorylation.

A further means to detect FAS phosphorylation is by the use of dephosphorylation to remove phosphate groups. The dephosphorylated polypeptides are not recognized by the antibody based methods described herein and so a loss of reactivity (or signal) would confirm an original phosphorylated state in a FAS molecule.

Additionally, dephosphorylated FAS polypeptides and fragments thereof may be used to screen for and/or produce antibodies specific for the phosphorylated polypeptide or fragment relative to the non-phosphorylated polypeptide or fragment thereof.

### Example 5

### Increased FAS Phosphorylation

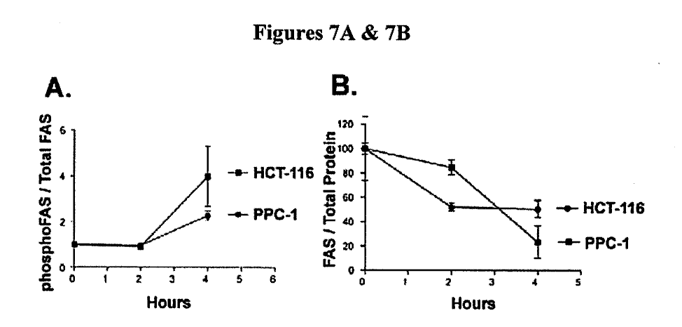

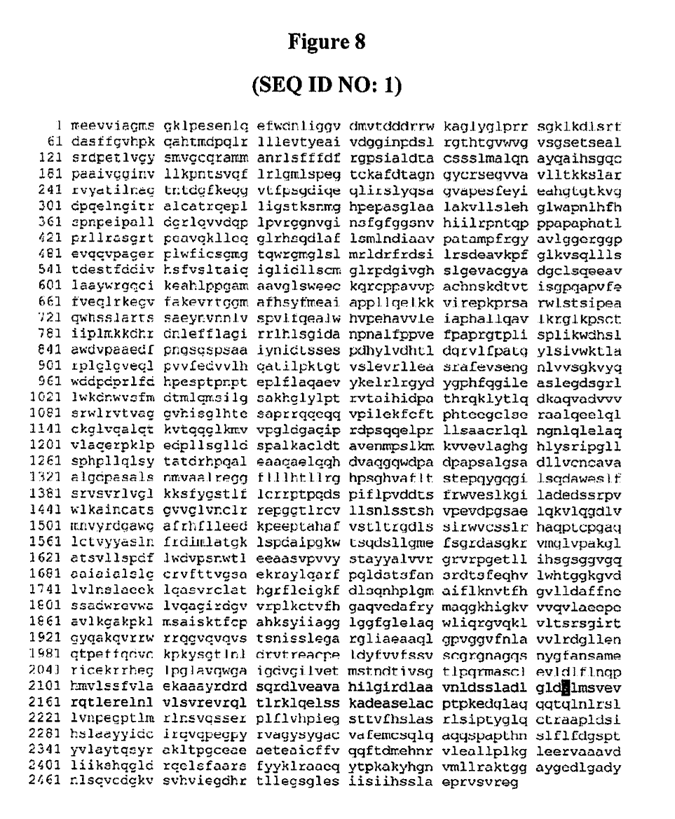

The effect of okadaic acid, a protein phosphatase 2A inhibitor, in FAS phosphorylation was studied. FIG. 7A shows results demonstrating that a brief treatment with 100 nM okadaic acid, a concentration that is specific for protein phosphatase 2A′ inhibition (Yan and Mumby, J. Biol. Chem., 274: 31917-31924 (1999)), the ratio of phosphorylated FAS to total FAS increases substantially in both PPC-1 and HCT-116 cell lines. FIG. 7 B shows that increased phosphorylation of FAS leads to a reduction in total cellular FAS.

So it is possible that FAS phosphorylation is linked to its degradation by one of two ways: direct dephosphorylation of FAS or by inactivation of the kinase(s) that phosphorylates FAS.

### REFERENCES

1. De Schrijver et al., Cancer Res., 63: 3799-3804 (2003)
2. Heiligtag et al., Cell Death Differ. 9: 1017-1025 (2002)
3. Kuhajda et al., Proc. Natl. Acad. Sci. USA, 91: 6379-6383 (1994)
4. Li et al., Cancer Res., 61: 1493-1499 (2001)
5. Pizer et al., Cancer Res., 56: 2745-2747 (1996)
6. Pizer et al., Cancer Res. 56: 1189-1193 (1996)
7. Pizer et al., Cancer Res. 56: 745-751 (1996)
8. Pizer et al., Cancer Res. 60:213-218 (2000)
9. Slade et al., Anticancer Res., 23: 1235-1243 (2003)
10. Pizer et al., Prostate 47: 102-110 (2001)
11. Zhou et al., Cancer Res., 63:7330-7337 (2003)
12. Wang et al., Cancer Lett., 167: 99-104 (2001)
13. Wang et al, Clin. Chim. Acta., 304: 107-115 (2001)
14. Wang et al., J. Immunoassay Immunochem. 23: 279-292 (2002)
15. Wang et al., J. Exp. Ther. Oncol., 4: 101-110 (2004)
16. Alo et al., Cancer, 77: 474-482 (1996)
17. Alo et al., Tumori, 85: 35-40 (1999)
18. Alo et al., Am. J. Clin. Pathol., 116: 129-134 (2001)
19. Buchsbaum et al., Immunogenetics, 55:172-176 (2003)
20. Milgraum et al., Clin. Cancer Res., 3: 2115-2120 (1997)
21. Nakamura et al., Int. J. Mol. Med., 4: 381-387 (1999)
22. Bull et al., Br. J. Cancer, 84: 1512-1519 (2001)
23. Epstein et al., Urology, 45: 81-86 (1995)
24. Shurbaji et al., Hum. Pathol, 27: 917-921 (1996)
25. Swinnen et al., Int. J. Cancer, 98: 198-22 (2002)
26. Baron et al., J. Cell. Biochem., 91: 47-53 (2004)
27. Rossi et al., Mol. Cancer. Res., 1: 707-715 (2003)
28. Shah et al., Hum. Pathol., 37: 401-409 (2006)
29. Welsh et al., Cancer Res., 61: 5974-5978 (2001)
30. Rashid et al., Am. J. Pathol., 150: 201-208 (1997)
31. Visca et al., Clin. Cancer Res., 5: 4111-4118 (1999)
32. Ogino et al., Mod. Pathol, 19: 59-68 (2006)
33. Kusakabe et al., Histopathology, 40: 71-79 (2002)
34. Piyathilake et al., Hum. Pathol., 31: 1068-1073 (2000)
35. Wang et al.,
36. Visca et al., Anticancer Res., 24: 4169-4173 (2004)
37. Gabrielson et al., Clin. Cancer Res., 7: 153-157 (2001)
38. Krontiras et al., Head Neck, 21: 325-329 (1999)
39. Innocenzi et al., J. Cutan. Pathol., 30: 23-28 (2003)
40. Nemoto et al., Pathobiology 69:297-303 (2001)
41. Silva et al., Oral Oncol, 40: 688-696 (2004)
42. Alo et al., Oncol. Rep. 7:1383-1388 (2000)
43. Gansler et al., Hum. Pathol. 28: 686-692 (1997)
44. Kapur et al., Mod. Pathol. 18: 197-204 (2005)
45. Pizer et al., Cancer, 83: 528-537 (1998)
46. Sebastiani et al., Gynecol. Oncol. 92: 101-105 (2004)
47. Tsuji et al., Acta Obstet. Gynecol. Scand., 83: 586-590 (2004)
48. Vlad et al., Modern Pathol., 12: 70A (1999)
49. Ricci et al., Ann. Diagn. Pathol., 6: 229-235 (2002)
50. Nicol et al., Moden Pathol. 16: 164A (2003)
51. Visca et al., Anticancer Res., 23:335-339 (2003)
52. Turyn et al., Horm. Metab. Res. 35: 565-569 (2003)
53. Camassei et al., Invest. Ophthalmol. Vis. Sci., 44: 2399-2403 (2003)
54. Camassei et al., Med. Pediatr. Oncol., 40: 302-308 (2003)
55. Takahiro et al., Clin. Cancer Res., 9: 2204-2212 (2003)
56. Smith, FASEB J, 8: 1248-1259 (1994)
57. Wakil, Biochem. 28: 4523-4530 (1989)
58. Ruderman et al., Am. J. Physiol., 276: E1-E18 (1999)
59. Weiss et al., Biol. Chem. Hoppe Seyler; 367:905-912 (1986)
60. Pizer et al., Int. J. gynecoL Pathol., 16: 45-51 (1997)
61. Smith et al., J. Natl. Cancer Inst., 73: 323-329 (1984)
62. Thompson et al., Pediatr. Res., 19: 139-143 (1985)
63. Ridsdale and Post, Am. J. Physiol. Lung Cell. Mol. Physiol., 287: L743-751 (2004)
64. Kim et al., Am. J. Physiol. Endocrinol. Metab., 283: E867-879 (2002)
65. Landree et al., J. Biol. Chem., 279: 3817-3827 (2004)
66. Jackson et al., J. Cell. Biol., 121: 317-333 (1993)
67. Duden, Mol. Membr. Biol., 20: 197-207 (2003)
68. Keon et al., Int. J. Biochem., 25: 533-543 (1993)
69. Hennigar et al., Biochim Biophys Acta, 1392: 85-100 (1998)
70. Rous, FEBS Lett., 44: 55-58 (1974)
71. Ramakrishna and Benjamin, Prep. Biochem., 13: 475-488 (1983)
72. Clark, J. Clin. Gastroenterol., 40 Suppl (1): S5-S10 (2006)
73. Brunt, Semin. Liver Dis., 21: 3-16 (2001)
74. Spiegelman and Flier, Cell, 104: 531-543 (2001)
75. Rapp et al., Br. J. Cancer, 93: 1062-1067 (2005)
76. Lukanova et al., Int. J. Cancer, 118: 458-466 (2006)
77. Dignam et al., Breast Cancer Res. Treat. 1-10 (2005)
78. Calle et al., N. Engl. J. Med., 348: 1625-1638 (2003)
79. Petrelli et al., Cancer Causes Control, 13: 325-332 (2002)
80. Morimoto et al., Cancer Causes Control, 13: 974-951 (2002)
81. Haglund et al., Biochem. J., 357: 437-445 (2001)
82. Nilsson-Ekdahl and Nilsson, Eur. J. Immunol., 31: 1047-1054 (2001)
83. Labugger et al., Circulation 102: 1221-1226 (2000)
84. Luftner et al., Anticancer Res., 23: 991-997 (2003)
85. Goldknopf, et al., Biochem. Biophys. Res. Commun., 342: 1034-1039 (2006)
86. Oetting et al., J. Biol. Chem., 261: 1649-1652 (1986)
87. Shaw et al., Biochem. Biophys. Res. Commun., 336: 1268-1277 (2005)
88. Thompson et al., Biochem. Biophys Acta, 662: 125-130 (1981)
89. Yan and Mumby, J. Biol. Chem., 274: 31917-31924 (1999)
90. Rechsteiner and Rogers, Trends Biochem. Sci., 21: 267-271 (1996)
91. Graner et al., Cancer Cell, 5: 253-261 (2004)
92. Joshi et al., J. Biol. Chem., 278: 33142-33149 (2003)
93. Goodman et al., Electrophoresis, 25: 2533-2538 (2004)
94. Schulenberg et al., Electrophoresis, 25: 2526-2532 (2004)
95. Loyet et al., Mol. Cell. Proteomics, 4: 235-245 (2005)
96. Hayduk et al., Electrophoresis, 25: 2545-2556 (2004)
97. Carr et al., Methods Enzymol., 405: 82-115 (2005)
98. Shenolikar and Ingebritsen, Methods Enzymol., 107: 102-129 (1984)
99. Swarup et al., J. Biol. Chem., 256: 8197-8201 (1981)
100. Shenolikar, Annu. Rev. Cell. Biol., 10: 55-86 (1994).

All references cited herein, including patents, patent applications, and publications, are hereby incorporated by reference in their entireties, whether previously specifically incorporated or not.

Having now fully described the inventive subject matter, it will be appreciated by those skilled in the art that the same can be performed within a wide range of equivalent parameters, concentrations, and conditions without departing from the spirit and scope of the disclosure and without undue experimentation.

While this disclosure has been described in connection with specific embodiments thereof, it will be understood that it is capable of further modifications. This application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth.

1-12. (canceled)

13. A method of identifying the presence of cancer in a subject, said method comprising:
detecting the presence of a fatty acid synthase (FAS) polypeptide comprising a phosphorylated threonine residue represented by position 1982 in SEQ ID NO:1 or by position 1984 in SEQ ID NO:2, or a fragment of said polypeptide comprising said phosphorylated threonine residue, in a biological sample from said subject.

14. A method of monitoring cancer burden in a subject, said method comprising
measuring the level of a fatty acid synthase (FAS) polypeptide comprising a phosphorylated threonine residue represented by position 1982 in SEQ ID NO:1 or by position 1984 in SEQ ID NO:2, or a fragment of said polypeptide comprising said phosphorylated threonine residue, in a biological sample from said subject; and
repeating said measuring over time, wherein an increase or decrease in the level over time indicates an increase or decrease, respectively, in cancer burden.

15. The method of claim 13, wherein said FAS polypeptide comprising one or more phosphorylated amino acid residue has a relative molecular weight of about 270 kDa and a length of 2509 amino acid residues or less, or 2511 amino acid residues or less.

16. The method of claim 13, wherein said FAS polypeptide fragment has a length of at least 5 amino acid residues and comprises said phosphorylated threonine residue.

17. The method of claim 13, wherein said biological sample is selected from a blood sample, a serum sample, a plasma sample or a tumor cell containing sample.

18. The method of claim 13, wherein said cancer is breast cancer.

19. The method of claim 14, wherein said FAS polypeptide comprising one or more phosphorylated amino acid residue has a relative molecular weight of about 270 kDa and a length of 2509 amino acid residues or less, or 2511 amino acid residues or less.

20. The method of claim 14, wherein said FAS polypeptide fragment has a length of at least 5 amino acid residues and comprises said phosphorylated threonine residue.

21. The method of claim 14, wherein said biological sample is selected from a blood sample, a serum sample, a plasma sample or a tumor cell containing sample.

22. The method of claim 14, wherein said cancer is breast cancer.

23. An antibody that binds an epitope comprising a phosphorylated threonine in a fatty acid synthase (FAS) polypeptide, or in a fragment of said polypeptide comprising said phosphorylated threonine residue,
wherein said phosphorylated threonine residue is represented by position 1982 in SEQ ID NO:1 or by position 1984 in SEQ ID NO:2.

24. The antibody of claim 23, wherein said FAS polypeptide has a relative molecular weight of about 270 kDa and a length of 2509 amino acid residues or less, or 2511 amino acid residues or less.

25. The antibody of claim 23, wherein said FAS polypeptide fragment has a length of at least 5 amino acid residues and comprises said phosphorylated threonine residue.

26. The antibody of claim 23, wherein said antibody binds the epitope in a blood sample, a serum sample, a plasma sample or a tumor cell containing sample.

27. The antibody of claim 23, which is a monoclonal antibody.

28. The antibody of claim 23, wherein said antibody binds the epitope in a blood sample, a serum sample, a plasma sample or a tumor cell containing sample.
